(19) United States
(12) Patent Application Publication
(10) Pub. No.: US 2026/0041381 A1
(43) Pub. Date: Feb. 12, 2026

(54) BODY MOVEMENT DISPLAY APPARATUS, OPERATION METHOD OF BODY MOVEMENT DISPLAY APPARATUS, AND IMAGE DIAGNOSTIC SYSTEM

(71) Applicant: FUJIFILM Corporation, Tokyo (JP)

(72) Inventors: Akiko MORI, Tokyo (JP); Hisaaki OCHI, Tokyo (JP); Yukio KANEKO, Tokyo (JP)

(73) Assignee: FUJIFILM Corporation, Tokyo (JP)

(21) Appl. No.: 19/292,847

(22) Filed: Aug. 6, 2025

(30) Foreign Application Priority Data
Aug. 9, 2024 (JP) 2024-134615

(51) Int. Cl. A61B 6/04; A61B 6/00; A61B 6/46; G01R 33/56; G01R 33/565

(52) U.S. Cl. CPC A61B 6/0492; A61B 6/463; A61B 6/547; G01R 33/5608; G01R 33/56509

(57) ABSTRACT

Provided are a body movement display apparatus, an operation method thereof, and an image diagnostic system that reduce a movement of a line of sight of a subject and enable the subject to satisfactorily recognize his/her own body movement. A body movement display apparatus includes a processor that displays an image in a manner that is visible to a subject during an examination of the subject using an MRI apparatus, and a body movement detection sensor including a first camera and a second camera that detect a body movement of the subject, in which the processor generates a first graphic that is rotationally symmetric about a fixed center and whose size changes according to a magnitude of the body movement of the subject detected by the body movement detection sensor, and projects the generated first graphic into a bore of the MRI apparatus as the image using the projector.

### CROSS-REFERENCE TO RELATED APPLICATION

The present application claims priority under 35 U.S.C § 119(a) to Japanese Patent Application No. 2024-134615 filed on Aug. 9, 2024, which is hereby expressly incorporated by reference, in its entirety, into the present application.

### BACKGROUND OF THE INVENTION

### 1. Field of the Invention

The present invention relates to a body movement display apparatus, an operation method of a body movement display apparatus, and an image diagnostic system, and particularly relates to a technique that enables a subject to recognize his/her body movement satisfactorily.

### 2. Description of the Related Art

A magnetic resonance imaging (MRI) apparatus used for image diagnosis can non-invasively acquire information from the entire body of a living body, and thus is widely used in a medical field.

An MRI apparatus having such a feature takes a long time to perform one imaging session and is easily affected by a body movement of a subject during data acquisition, and a body movement of the subject during imaging affects image quality. Therefore, it is necessary to reduce the body movement of the subject during the data acquisition. In a case in which the subject is notified of his/her own body movement, the subject himself/herself pays attention to the body movement, which is expected to have an effect of reducing the body movement during the imaging.

As a method of notifying of the body movement of the subject, a notification method using a graphic has been proposed (JP2006-158762A).

In order to efficiently perform respiratory-gated imaging, the MRI apparatus disclosed in JP2006-158762A displays a respiratory state in a manner that can be visually recognized by the subject, and particularly displays a depth of breathing using gradations or a light-emitting position of a light spot. As a result, the subject can adjust his/her own respiratory state.

### SUMMARY OF THE INVENTION

In a case of the MRI apparatus disclosed in JP2006-158762A, the subject moves his/her line of sight in a case of checking the gradations or the light-emitting position of the light spot indicating the depth of breathing, which increases the likelihood that the movement of the line of sight induces a movement of a head and a movement of an imaging target part other than the head.

The present invention has been made in view of such circumstances, and an object of the present invention is to provide a body movement display apparatus, an operation method of a body movement display apparatus, and an image diagnostic system that suppress a movement of a line of sight of a subject and enable the subject to satisfactorily recognize his/her own body movement.

An invention according to a first aspect is a body movement display apparatus comprising: a processor; a display that displays an image in a manner that is visible to a subject during an examination of the subject using an image diagnostic apparatus; and a body movement detection sensor that detects a body movement of the subject, in which the processor generates a first graphic that is rotationally symmetric about a fixed center and whose size changes according to a magnitude of the body movement of the subject detected by the body movement detection sensor, and causes the display to display the generated first graphic as the image.

According to the first aspect of the present invention, the subject during the examination using the image diagnostic apparatus can check the magnitude of his/her own body movement by viewing the first graphic that is rotationally symmetric about the fixed center and whose size changes according to the magnitude of his/her own body movement, and since the center of the first graphic (the center of the rotational symmetry) does not move, the subject can fix the line of sight in a case of viewing the first graphic. That is, it is possible to further suppress the movement of the line of sight of the subject, and it is possible to reduce the induction of the movement of the head due to the movement of the line of sight and the movement of the imaging target part other than the head.

According to a second aspect of the present invention, in the body movement display apparatus according to the first aspect, it is preferable that the processor converts the magnitude of the body movement of the subject into the size of the first graphic, and generates the first graphic corresponding to the converted size of the first graphic.

According to a third aspect of the present invention, in the body movement display apparatus according to the second aspect, it is preferable that the conversion of the magnitude of the body movement of the subject into the size of the first graphic is a linear conversion or a non-linear conversion. In a case of the linear conversion, the size of the first graphic is changed in a certain relationship according to the magnitude of the body movement, and thus it is easy to understand as a method of displaying a state of the body movement. Meanwhile, in a case of the non-linear conversion, the first graphic can be displayed by increasing the sensitivity with respect to the magnitude of the body movement for which it is desired to further suppress the body movement, thereby encouraging the subject to adjust the body movement.

According to a fourth aspect of the present invention, in the body movement display apparatus according to the second or third aspect, it is preferable that the conversion of the magnitude of the body movement of the subject into the size of the first graphic is a conversion weighted based on a magnitude of an influence of the body movement of the subject on imaging of the image diagnostic apparatus.

According to a fifth aspect of the present invention, in the body movement display apparatus according to the fourth aspect, it is preferable that the magnitude of the influence of the body movement of the subject on the imaging of the image diagnostic apparatus changes depending on at least one of an imaging target part of the subject imaged by the image diagnostic apparatus, an imaging sequence in the image diagnostic apparatus, or a k-space filling method used in the image diagnostic apparatus. In a case of converting the magnitude of the body movement of the subject into the size of the first graphic, the conversion is weighted in consideration of imaging conditions such as the imaging target part, the imaging sequence, and the k-space filling method, thereby making it possible to perform a conversion suitable for the state display of the body movement that affects the imaging.

According to a sixth aspect of the present invention, in the body movement display apparatus according to any one of the first to fifth aspects, it is preferable that a boundary value of the magnitude of the body movement of the subject that is permissible by the image diagnostic apparatus and that presents a relation with the size of the first graphic is set, and that the processor causes the display to display a second graphic having a size corresponding to the boundary value and having a similar outer shape to the first graphic such that a center of the second graphic coincides with the center of the first graphic. By displaying the second graphic having the size corresponding to the boundary value on the display, the subject can adjust the body movement so that the body movement does not exceed the second graphic. In addition, since the second graphic has the similar shape to the first graphic and has the center that coincides with the center of the first graphic, the first graphic and the second graphic can be visually recognized simultaneously without moving the line of sight.

According to a seventh aspect of the present invention, in the body movement display apparatus according to the sixth aspect, it is preferable that the boundary value is set by at least one of an imaging target part of the subject imaged by the image diagnostic apparatus, an imaging sequence in the image diagnostic apparatus, or a k-space filling method used in the image diagnostic apparatus. In a case in which the magnitude of the body movement of the subject that is permissible by the image diagnostic apparatus may change depending on the imaging conditions such as the imaging target part of the subject, the imaging sequence, and the k-space filling method, it is preferable to set the boundary value according to the imaging conditions.

According to an eighth aspect of the present invention, in the body movement display apparatus according to the sixth or seventh aspect, it is preferable that the processor causes the display to display the first graphic and the second graphic such that the first graphic and the second graphic differ in at least one of a color, a line type, or brightness.

According to a ninth aspect of the present invention, in the body movement display apparatus according to any one of the sixth to eighth aspects, it is preferable that the processor issues a warning in a case in which the magnitude of the body movement of the subject approaches the boundary value beyond a threshold value or exceeds the boundary value. As a result, it is possible to further encourage the subject to suppress the body movement.

According to a tenth aspect of the present invention, in the body movement display apparatus according to the ninth aspect, it is preferable that the warning is issued by one or more of a warning sound generator, the display, an illumination device in a gantry of the image diagnostic apparatus, and a vibration generator.

According to an eleventh aspect of the present invention, in the body movement display apparatus according to any one of the first to tenth aspects, it is preferable that the body movement detection sensor includes a camera that images the subject and that outputs an image of the subject, and an image processing unit that processes the image to detect the body movement of the subject, and that the image processing unit extracts an imaging target part of the subject included in the image, the imaging target part being imaged by the image diagnostic apparatus, acquires a movement of the extracted imaging target part between consecutive frames of the image as a body movement vector of the imaging target part, and detects the magnitude of the body movement of the subject from the body movement vector.

According to a twelfth aspect of the present invention, in the body movement display apparatus according to any one of the first to eleventh aspects, it is preferable that the first graphic has an outer shape of a circle or a regular polygon.

An invention according to a thirteenth aspect is an image diagnostic system comprising: an image diagnostic apparatus; and the body movement display apparatus according to any one of the first to twelfth aspects.

According to a fourteenth aspect of the present invention, in the image diagnostic system according to the thirteenth aspect, it is preferable that the image diagnostic apparatus includes a magnetic resonance imaging apparatus or an X-ray CT apparatus.

An invention according to a fifteenth aspect is an operation method of a body movement display apparatus including a processor, a display that displays an image in a manner that is visible to a subject during an examination of the subject using an image diagnostic apparatus, and a body movement detection sensor that detects a body movement of the subject, the operation method comprising: a step of, via the processor, acquiring a magnitude of the body movement of the subject from the body movement detection sensor; a step of, via the processor, generating a first graphic that is rotationally symmetric about a fixed center and whose size changes according to the acquired magnitude of the body movement of the subject; and a step of, via the processor, causing the display to display the generated first graphic as the image.

According to the present invention, the subject during the examination using the image diagnostic apparatus can view the first graphic that is rotationally symmetric about the fixed center and whose size changes according to the magnitude of his/her own body movement, and the subject can recognize his/her own body movement satisfactorily. In particular, by viewing the first graphic that is rotationally symmetric about the fixed center, it is possible to further suppress the movement of the line of sight of the subject, and it is possible to reduce the induction of the movement of the head due to the movement of the line of sight and the movement of the part other than the head.

### BRIEF DESCRIPTION OF THE DRAWINGS

FIG. 1 is a perspective view showing an appearance of a magnetic resonance imaging apparatus (MRI apparatus) to which a respiratory motion display device according to the present invention is applied.

FIG. 2 is a diagram showing a schematic configuration of an inside of the MRI apparatus shown in FIG. 1.

FIG. 3 is a diagram showing an external configuration of a main part of a body movement display apparatus according to an aspect of the present invention.

FIG. 4 is a block diagram showing an embodiment of an image diagnostic system according to the present invention.

FIG. 5 is a diagram showing an example of an image projected by a projector, and is a diagram particularly showing an image in which a first graphic C1 and a second graphic C2 are combined.

FIG. 6 is a graph showing an example of a relationship between a magnitude of a body movement of a subject and a size of an object (first graphic).

FIG. 7 is a graph showing another example of a relationship between a magnitude of a body movement of the subject and a size of the object (first graphic).

FIG. 8 is a diagram showing a relationship between a change in size of the object (first graphic) and a change in object display presented to the subject, the change in size of the object being associated with a change in body movement of the subject.

FIG. 9 is a flowchart showing an embodiment of an operation method of the body movement display apparatus according to the aspect of the present invention.

FIG. 10 is a diagram showing a part of a graphic projected by the projector, and is a diagram particularly showing a mark for fixing a line of sight.

FIG. 11 is a diagram showing a graphic obtained by combining a first graphic H1 and a second graphic H2.

### DESCRIPTION OF THE PREFERRED EMBODIMENTS

Hereinafter, preferred embodiments of a body movement display apparatus, an operation method of a body movement display apparatus, and an image diagnostic system according to the present invention will be described with reference to the accompanying drawings.

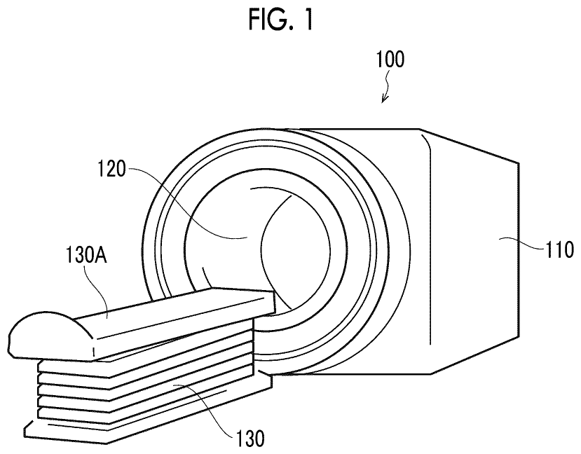

FIG. 1 is a perspective view showing an appearance of a magnetic resonance imaging apparatus (MRI apparatus) to which a body movement display apparatus according to the embodiment of the present invention is applied.

An MRI apparatus 100 shown in FIG. 1 comprises a gantry 110 and an examination bed 130 comprising a top plate 130A disposed on a front side of a bore 120, which is a cylindrical imaging space provided in the gantry 110.

### Internal Configuration of MRI Apparatus

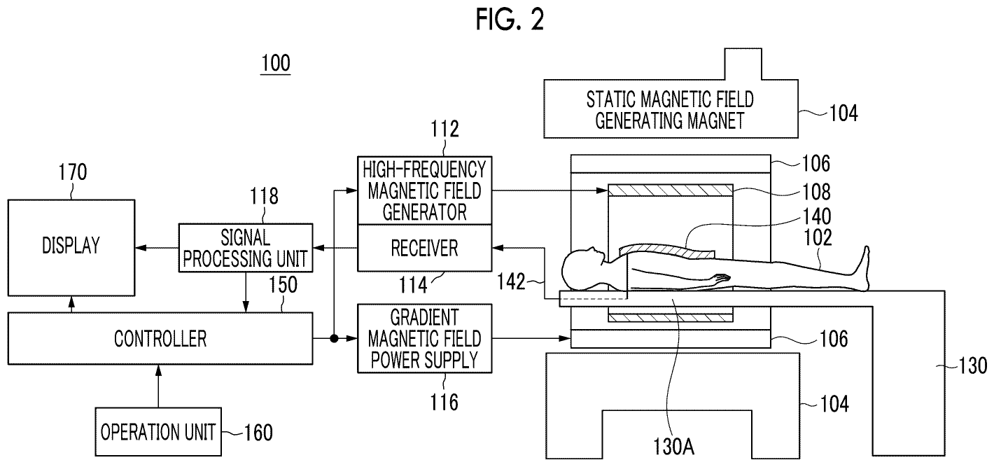

FIG. 2 is a diagram showing a schematic configuration of an inside of the MRI apparatus shown in FIG. 1.

As shown in FIG. 2, the MRI apparatus 100 comprises a static magnetic field generating magnet 104 that generates a uniform static magnetic field in an imaging space in which a subject 102 is placed, a gradient magnetic field coil (GC coil) 106, a radio frequency (RF) coil (transmission coil) 108, a receive coil 140, a high-frequency magnetic field generator 112, a receiver 114, and a gradient magnetic field power supply 116.

The gradient magnetic field coil 106 is composed of gradient magnetic field coils in three directions of X, Y, and Z, and generates a gradient magnetic field pulse in the imaging space in response to a signal from the gradient magnetic field power supply 116. The transmission coil 108 generates a high-frequency magnetic field that causes a nuclear magnetic resonance signal (NMR signal) to be generated in a nucleus of an atom constituting a tissue of the subject 102 in response to a signal from the high-frequency magnetic field generator 112.

The receive coil 140 detects the NMR signal generated from the subject 102. The detected NMR signal is transmitted to the receiver 114 via a signal cable 142. The signal is subjected to analog-digital (AD) conversion using an AD converter in the receiver 114 to generate measurement data (raw data).

In addition, the MRI apparatus 100 further comprises a signal processing unit 118, a controller 150, an operation unit 160, and a display 170.

The signal processing unit 118 performs inverse Fourier transform on the measurement data generated by the receiver 114 to reconstruct an image, and outputs the reconstructed image signal to the controller 150 and the display 170.

In FIG. 2, an example in which the receive coil 140 is connected to the signal processing unit 118 and the controller 150 via the signal cable 142 has been described, but the connection between the receive coil 140 and the signal processing unit 118 and/or the controller 150 is not limited to wired and may be wireless. As an example of the wireless connection, the receive coil 140 includes an AD converter and a wireless communication module, and digital data (for example, measurement data) generated by the receive coil 140 is wirelessly transmitted to the wireless communication module in the signal processing unit 118 and/or the controller 150.

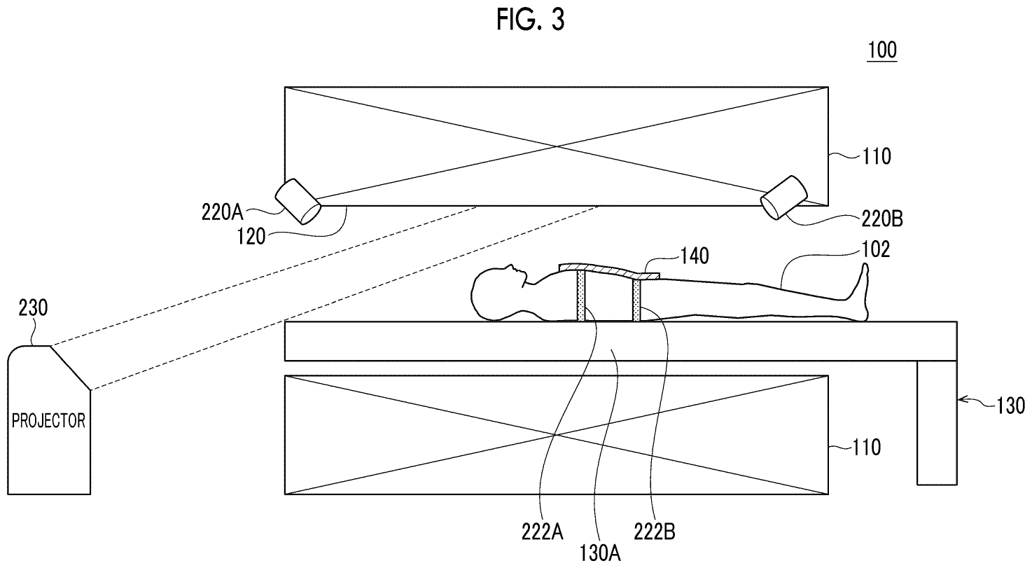

The controller 150 has a measurement controller and a calculation unit (not shown) and controls the entire apparatus including the high-frequency magnetic field generator 112, the gradient magnetic field power supply 116, and the display 170. The display 170 displays the reconstructed image and an image of the subject 102 captured by a first camera 220A and a second camera 220B shown in FIG. 3, and functions as a part of a user interface in a case in which an operator inputs various parameters and the like.

The controller 150 sends commands to the high-frequency magnetic field generator 112 and the gradient magnetic field power supply 116 according to an imaging target part (position and size of a specific region) of the subject and an imaging sequence (protocol of the examination (imaging plan) and pulse sequence according to the imaging plan), which are set by the operator operating the operation unit 160, and generates the high-frequency magnetic field and the gradient magnetic field.

There are a plurality of k-space filling methods of filling a k-space (Fourier space) with data necessary for image reconstruction, and the k-space filling method is also set appropriately. Details of the k-space filling method will be described below.

In addition, the controller 150 generates a file of a format for a medical image from an image signal designated by the operation unit 160 among the image signals processed by the signal processing unit 118, and registers the file in an image database (not shown) or the like.

The signal processing unit 118 and the controller 150 can be realized by, for example, a computer comprising a processor such as a central processing unit (CPU) and a memory that stores a control program, a parameter, and the like, executing a calculation or a control program.

FIG. 3 is a diagram showing an external configuration of a main part of the body movement display apparatus according to the embodiment of the present invention. In FIG. 3, parts common to those in FIGS. 1 and 2 are denoted by the same reference numerals, and detailed description thereof will be omitted.

In FIG. 3, a first camera 220A and a second camera 220B that image the subject 102 in the bore 120 are disposed in the gantry 110. The first camera 220A and the second camera 220B function as a part of a body movement detection sensor that detects a body movement of the subject 102.

That is, the body movement detection sensor of this example comprises the first camera 220A and the second camera 220B, and an image processing unit that detects a body movement of an imaging target part of the subject 102 from the image captured by at least one of the first camera 220A or the second camera 220B.

The image processing unit extracts an imaging target part of the subject 102 included in the image captured by at least one of the first camera 220A or the second camera 220B, the imaging target part being imaged by the MRI apparatus 100, acquires a movement of the extracted imaging target part between consecutive frames of the image as a body movement vector (displacement vector) of the imaging target part, and detects a magnitude of the body movement of the subject from the acquired body movement vector.

In addition, in a case in which the imaging target part is the chest and/or the abdomen, respiratory bands 222A and 222B worn on the chest and/or the abdomen of the subject 102 function as a part of a body movement detection sensor for detecting the body movement (respiratory motion) of the chest and/or the abdomen of the subject 102.

The number of the cameras is not limited to two, and may be one or three or more. In addition, an installation position of the camera is not limited to diagonally above the subject or to the bore 120. Further, the camera is not limited to a visible light camera, and can be, for example, an infrared camera.

In addition, the body movement detection sensor may be a sheet in which a plurality of pressure sensors to be placed under the subject are incorporated. Body movement information of the subject can be obtained from a pressure signal detected by the pressure sensor in the sheet in response to a motion of the subject 102.

A projector 230 projects an image into the bore 120 and functions as a display that displays the image in a manner that is visible to the subject 102 during the examination of the subject 102.

### Image Diagnostic System

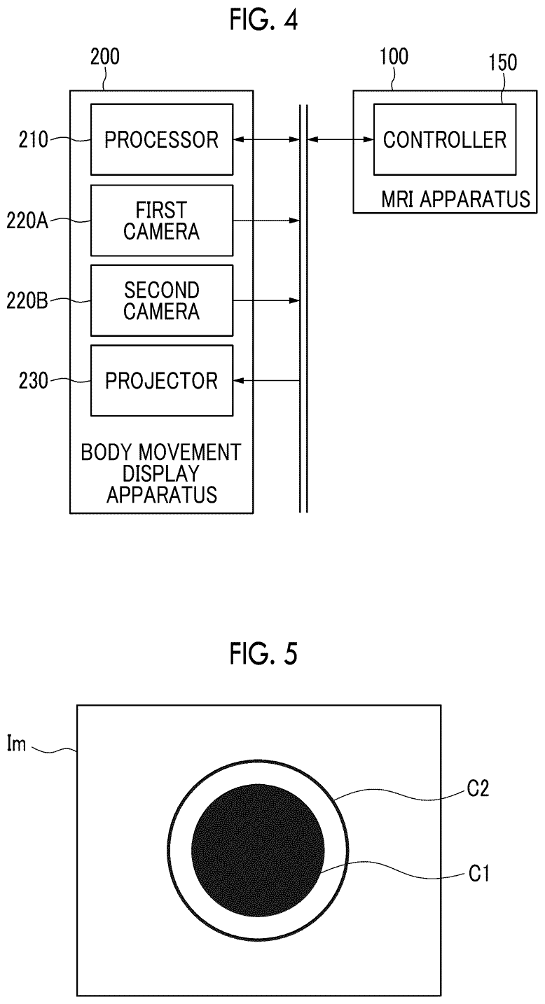

FIG. 4 is a block diagram showing an embodiment of an image diagnostic system according to the present invention.

The image diagnostic system shown in FIG. 4 comprises the MRI apparatus 100 which is an image diagnostic apparatus, and a body movement display apparatus 200. The configuration of the MRI apparatus 100 is specifically shown in FIGS. 1 and 2.

The body movement display apparatus 200 is composed of a processor 210, the first camera 220A, the second camera 220B, and the projector 230.

The first camera 220A and the second camera 220B may be cameras that are originally provided in the MRI apparatus 100 for imaging the state of the subject 102. In addition, instead of the first camera 220A and the second camera 220B, the respiratory bands 222A and 222B and other body movement detection sensors may be used.

The processor 210 is composed of a CPU or the like, performs overall control of each unit of the body movement display apparatus 200, and executes various kinds of processing including processing of generating an image projected from the projector 230.

The processor 210 and the controller 150 of the MRI apparatus 100 can communicate with each other, and the processor 210 causes the projector 230 to project an image showing the body movement of the subject 102 into the bore 120 during the examination of the MRI apparatus 100.

In a case in which the signal processing unit 118 and the controller 150 of the MRI apparatus 100 are composed of a computer comprising a processor and a memory as described above, the processor of the MRI apparatus 100 may function as the processor 210 of the body movement display apparatus 200.

### First Embodiment of Body Movement Display Apparatus

Next, a first embodiment of the body movement display apparatus according to the present invention will be described.

In a case in which the subject 102 enters an examination room in which the MRI apparatus 100 is installed and lies on the examination bed 130 of the MRI apparatus 100, the top plate 130A of the examination bed 130 is controlled such that the imaging target part of the subject 102 is positioned at the center of an imaging region in the bore 120, and then the MRI apparatus 100 starts imaging the imaging target part of the subject 102 in accordance with the imaging sequence.

The processor 210 of the body movement display apparatus 200 shown in FIG. 4 acquires an image captured by at least one of the first camera 220A or the second camera 220B during imaging (examination) of the MRI apparatus 100.

The processor 210 analyzes the acquired image of the subject 102 and extracts an image showing the imaging target part of the subject 102 or an image showing the imaging target part and its surrounding region. For example, in a case in which the imaging target part of the subject 102 is the abdomen, an image of the abdomen of the subject 102 or an image of the abdomen and its surrounding region is extracted from the image captured by at least one of the first camera 220A or the second camera 220B. In addition, in a case in which the imaging target part of the subject 102 is the head, an image of the head of the subject 102 or an image of the head and its surrounding region is extracted from the image captured by at least one of the first camera 220A or the second camera 220B. In a case in which the imaging target part is the head, it goes without saying that a receive coil that detects the NMR signal generated from the head of the subject 102 is used. The imaging target part of the subject 102 can be acquired from examination information of the subject 102.

Subsequently, the processor 210 acquires the body movement vector of the imaging target part by using an optical flow of the extracted image. That is, the processor 210 acquires a displacement vector of the imaging target part between adjacent frames of the image or a displacement vector of the imaging target part and its surrounding region as the body movement vector (body movement information).

Then, the processor 210 detects the magnitude of the body movement of the subject from the body movement vector. Specifically, the magnitude of the body movement of the subject is detected as an integrated value (area) of the body movement vector for a certain period of time. The certain period of time can be set to about a period of time during which the NMR signal is received within a time to repeat (TR) period, but is not limited to this and can be set as appropriate.

In this example, the processor 210 functions as an image processing unit that analyzes the acquired image of the subject 102 and that detects the magnitude of the body movement of the subject, but an image processing unit different from the processor 210 may acquire the image of the subject 102 from at least one of the first camera 220A or the second camera 220B and analyze the acquired image to detect the magnitude of the body movement of the subject. In addition, it is preferable that the magnitude of the body movement of the subject is continuously detected for each frame of the acquired image.

The processor 210 generates an object (first graphic C1) that is rotationally symmetric about the fixed center and whose size changes according to the detected magnitude of the body movement of the subject 102, and causes the projector 230 to project the generated first graphic C1 into the bore 120 as an image (see FIG. 5).

That is, the processor 210 converts the magnitude of the body movement of the subject into the size of the first graphic C1 and generates the first graphic C1 corresponding to the converted size of the first graphic C1. Details of the conversion of the magnitude of the body movement of the subject into the size of the first graphic C1 will be described below.

FIG. 5 is a diagram showing an example of an image projected by a projector, and is a diagram particularly showing an image in which a first graphic C1 and a second graphic C2 are combined.

A boundary value that is a boundary value of the magnitude of the body movement of the subject 102 that is permissible by the MRI apparatus 100 and that presents a relation with the size of the first graphic is set. Here, the boundary value is preferably set as a value of the magnitude of the body movement (affecting imaging) at a level that may result in body movement artifacts. Therefore, in a case in which the magnitude of the body movement of the subject 102 does not exceed the boundary value, the image quality of the image captured by the MRI apparatus 100 is acceptable.

The second graphic C2 is a graphic having a size corresponding to the boundary value and having a similar outer shape to the first graphic C1, and is a circle in this example.

The processor 210 can acquire the second graphic C2 from the memory in the processor 210 or an external memory, and generates an image Im to be projected by combining the generated first graphic C1 and the acquired second graphic C2.

The size of the image Im shown in FIG. 5 (the size of a projection region projected into the bore 120 from the projector 230) can be, for example, about 20 cm×30 cm.

In a case in which the size of the image Im is 20 cm×30 cm, the diameter of the second graphic C2 can be set to about 15 cm. Although it is common for the subject to remove their glasses during MRI imaging, even the subject with poor eyesight who cannot wear glasses can satisfactorily visually recognize the second graphic C2 having the above size.

It is preferable that the first graphic C1 included in the image Im generated by the processor 210 differs from the second graphic C2 in at least one of a color, a line type, or brightness. The first graphic C1 shown in FIG. 5 is filled with a color and/or brightness different from that of the second graphic C2.

Since the subject 102 shown in FIG. 3 is in a supine position, the image projected from the projector 230 is projected onto a ceiling of the bore 120 so that the subject 102 can visually recognize the image. However, in a case in which the subject is positioned in a lateral decubitus posture, it is preferable to project the image onto a side surface in the bore 120 so that the subject in a lateral decubitus posture can visually recognize the image. That is, it is preferable that the processor 210 projects the image onto a position in the bore 120 where the subject can easily visually recognize the image based on the information on the posture of the subject.

First Embodiment of Conversion of Magnitude of Body Movement of Subject into Size of First Graphic

The processor 210 converts the magnitude of the body movement of the subject into the size of the first graphic C1 and generates the first graphic C1 corresponding to the converted size of the first graphic C1. Therefore, in a case in which the magnitude of the body movement of the subject is large, the generated first graphic C1 is also large.

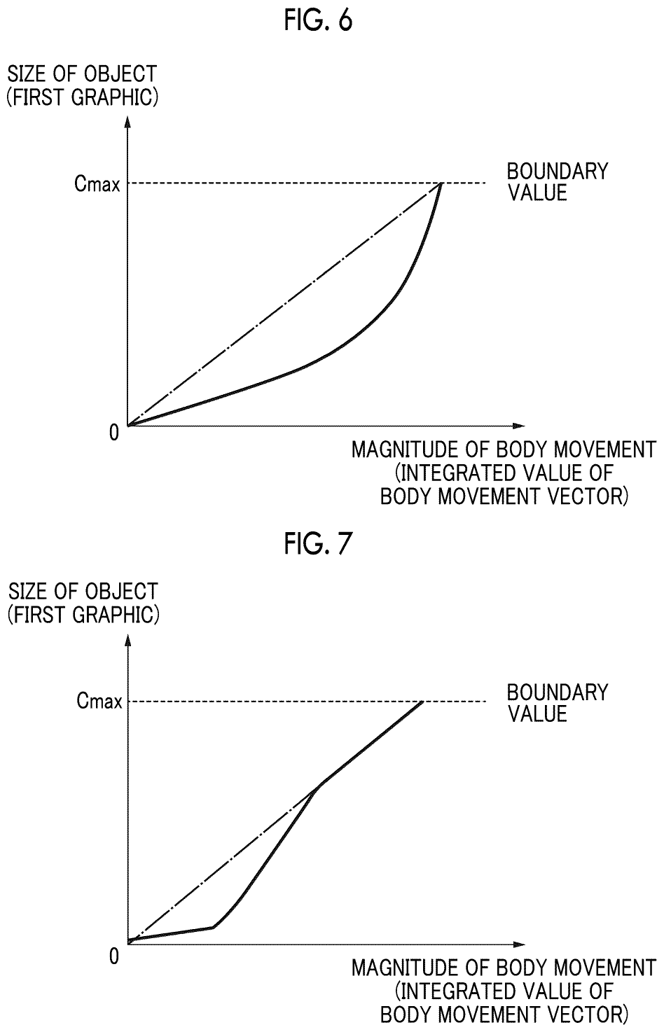

FIG. 6 is a graph showing an example of a relationship between a magnitude of a body movement of a subject and a size of an object (first graphic).

The processor 210 detects the magnitude of the body movement of the subject from the image in which the subject is captured, and, in a case in which the detected magnitude of the body movement is converted into the size (the diameter of the circle or the area of the circle) of the object (first graphic C1), the processor 210 performs linear conversion by the parameter indicated by a one-dot chain line graph in FIG. 6 or performs non-linear conversion by the parameter indicated by a solid line graph in FIG. 6.

Here, in a case in which the magnitude of the body movement of the subject (the integrated value of the body movement vector for a certain period of time) is a boundary value that permissible by the MRI apparatus 100, assuming that the size (the diameter or the area) of the first graphic is Cmax, the processor 210 performs linear conversion or non-linear conversion such that the size of the first graphic changes in a range of 0 to Cmax in a case in which the magnitude of the body movement of the subject changes in a range of 0 to the boundary value.

In a case of the linear conversion, the size of the first graphic C1 shown in FIG. 5 is changed in a certain relationship according to the magnitude of the body movement, and thus it is easy to understand as a method of displaying the state of the body movement. Meanwhile, in a case of the non-linear conversion, the first graphic C1 can be displayed by increasing the sensitivity with respect to the magnitude of the body movement for which it is desired to further suppress the body movement, thereby encouraging the subject to adjust the body movement.

In a case of the solid line graph showing the non-linear conversion in FIG. 6, as the magnitude of the body movement approaches the boundary value, the change in the size of the first graphic becomes steeper (the sensitivity becomes higher).

FIG. 7 is a graph showing another example of a relationship between a magnitude of a body movement of the subject and a size of the object (first graphic).

In a case of a solid line graph showing the non-linear conversion in FIG. 7, in a range where the magnitude of the body movement is small, the change in the size of the first graphic is small (the sensitivity is low) with respect to the change in the magnitude of the body movement, in a range where the magnitude of the body movement is intermediate, the change in the size of the first graphic is steep (the sensitivity is high) with respect to the change in the magnitude of the body movement, and in a range where the magnitude of the body movement is large, the change in the size of the first graphic with respect to the change in the magnitude of the body movement substantially coincides with a case of a one-dot chain line graph showing the linear conversion.

In a case of the solid line graph showing the non-linear conversion in FIG. 6, as the magnitude of the body movement approaches the boundary value, the change in the size of the first graphic becomes steeper, thereby making it easier for the subject to perceive his/her movement and attempt to stop. Meanwhile, in a case of the solid line graph showing the non-linear conversion in FIG. 7 (in a case in which the change in the first graphic becomes steep in the middle), the first graphic is displayed large even in a case in which the body movement is not as large as the magnitude of the body movement near the boundary value, so that the subject can always be conscious of not moving.

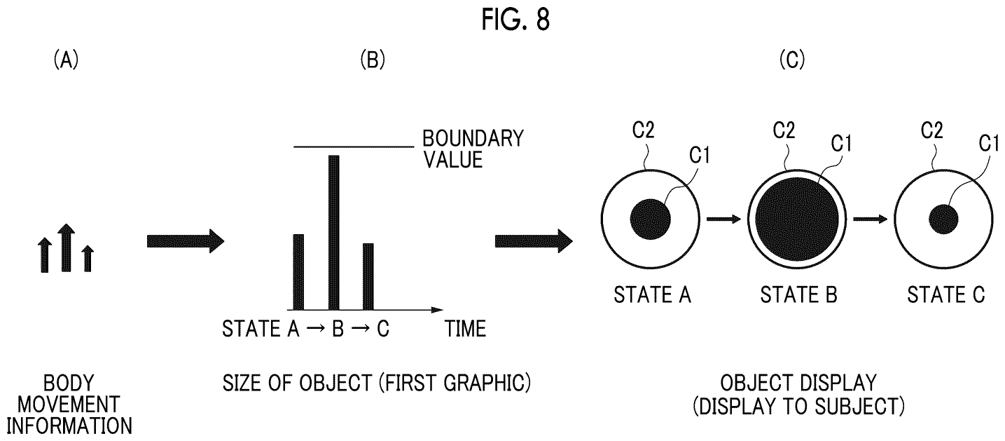

FIG. 8 is a diagram showing a relationship between a change in size of the object (first graphic) and a change in object display presented to the subject, the change in size of the object being associated with a change in body movement of the subject.

In FIG. 8, the processor 210 converts the body movement information into the size of the object (first graphic C1) ((A) and (B) of FIG. 8). In (B) of FIG. 8, the sizes of the first graphic C1 in states A, B, and C are shown by bar graphs.

Here, the state A indicates a state before the body movement becomes large, the state B indicates a state in which the body movement becomes large after the state A, and the state C indicates a state in which the body movement becomes small after the state B.

The processor 210 generates the first graphic C1 corresponding to the sizes of the first graphic C1 in the states A, B, and C shown in (B) FIG. 8, and combines the generated first graphic C1 and the second graphic C2 having the size corresponding to the boundary value to generate images in the states A, B, and C. Then, the processor 210 causes the projector 230 to project the generated images (images in the states A, B, and C, and the like) into the bore 120 ((C) of FIG. 8).

The subject 102 can observe the images of the object (first graphic C1) displayed in the state A→the state B→the state C during the examination of the MRI apparatus 100 ((C) of FIG. 8). In a case in which the subject observes the state A of the object display and in a case in which there is a body movement of the imaging target part, the object display transitions to the state B. The subject adjusts the body movement so as not to move the imaging target part by viewing the object display. Then, in a case in which the movement of the subject is suppressed and the object display transitions from the state B to the state C, the subject can check that the movement of the imaging target part is reduced by viewing the object display in the state C.

The object displays of the states A, B, and C shown in FIG. 8 are shown in relation to cases in which the body movement of the subject 102 changes, but the image actually projected from the projector 230 into the bore 120 is a moving image that can continuously change according to the movement of the body movement, and, for example, it is preferable that the image is a moving image that continuously changes in accordance with a frame rate (30 frames/second or 60 frames/second) of the moving image.

The subject 102 can grasp the magnitude of his/her own body movement in real time by viewing the image projected into the bore 120, and can suppress the body movement as necessary. In addition, the first graphic C1 representing the magnitude of the body movement in the image Im is a graphic (in this example, the outer shape is a circle) that is rotationally symmetric about the fixed center, changes only in size, and does not move. Therefore, the subject 102 can visually recognize the display of the first graphic C1 without moving his/her line of sight, thereby minimizing the movement of the eyes accompanied by the movement of the eyes and reducing the induction of the movement of the head and the movement of parts other than the head.

In addition to the first graphic C1 representing the magnitude of the body movement, the second graphic C2 representing the magnitude of the boundary value is displayed in a concentric circular shape with the first graphic C1, thereby allowing the subject 102 to compare the first graphic C1 and the second graphic C2. As a result, the subject 102 can grasp the magnitude of his/her current body movement, and can suppress the body movement so that the first graphic C1 does not exceed the second graphic C2.

Second Embodiment of Conversion of Magnitude of Body Movement of Subject into Size of First Graphic

The magnitude of the body movement of the subject 102 and the magnitude of the influence of the body movement of the subject 102 on the imaging of the MRI apparatus 100 do not necessarily correspond one-to-one.

That is, the magnitude of the influence of the body movement of the subject 102 on the imaging of the MRI apparatus 100 changes depending on, for example, the imaging target part of the subject by the MRI apparatus 100, the imaging sequence, and the k-space filling method, and, for example, even in a case in which the magnitude of the body movement of the subject 102 is the same, the influence on the imaging of the MRI apparatus 100 changes depending on the imaging target part of the subject 102, the imaging sequence, or the k-space filling method.

Therefore, it is preferable that the processor 210 performs a conversion weighted based on the magnitude of the influence of the body movement of the subject 102 on the imaging of the MRI apparatus 100 in a case of converting the magnitude of the body movement of the subject 102 into the size of the first graphic C1.

The processor 210 acquires, from the MRI apparatus 100, imaging conditions such as the imaging target part of the subject 102, the imaging sequence, and the k-space filling method, determines a weight (a weight corresponding to the magnitude of the influence on the imaging of the MRI apparatus 100) corresponding to at least one of the imaging conditions in a case of converting the magnitude of the body movement of the subject 102 into the size of the first graphic C1, and performs a conversion based on the determined weight.

For example, since it is necessary to reduce the body movement of the imaging target part in a collection period of a low frequency region rather than a high frequency region of the k-space, it is preferable to perform weighting such that the size of the circular area of the first graphic C1 is changed more sensitively during a period in which signals in the low frequency region are collected. In addition, since the collection period of the k-space differs depending on the imaging sequence or the k-space filling method, it is preferable to determine the height of a spatial frequency at a certain timing based on the parameters of the imaging sequence or the k-space filling method.

Hereinafter, details will be described.

The k-space has a low frequency region and a high frequency region, and a signal having high power is present in the low frequency region. In a case in which there is a body movement during the signal collection in the low frequency region, artifacts (false images) may appear throughout the entire image in a case of Fourier inverse transformation. Therefore, it is more important not to move the imaging part during the signal collection in the low frequency region. Therefore, in a case in which the magnitude of the body movement is converted into the size of the first graphic C1, a conversion coefficient is weighted according to the height of the spatial frequency of the signal to be collected. As a result, the size (size of the circular area) of the first graphic C1 representing the body movement of the subject can be displayed while being changed in the size more sensitively with respect to the body movement during the collection period in the low frequency region of the k-space (than during the collection period in the high frequency region). In a case in which the body movement during a period in which artifacts are likely to occur is displayed with particular emphasis, the subject can be encouraged to further suppress the body movement during the period.

In addition, in the first embodiment of converting the magnitude of the body movement of the subject into the size of the first graphic, the magnitude of the body movement of the subject is linearly converted or non-linearly converted into the size of the first graphic C1 as shown in the graphs of FIGS. 6 and 7, but, in a second embodiment of converting the magnitude of the body movement of the subject into the size of the first graphic, it is preferable to perform the second embodiment together with the first embodiment.

For example, in a case of the graph shown by the one-dot chain line in FIG. 6, the linear conversion is performed in converting the magnitude of the body movement of the subject into the size of the first graphic C1, but, by weighting the parameters of the linear conversion according to the magnitude of the influence on the imaging, the slope of the graph shown by the one-dot chain line in FIG. 6 can be changed, and thus, the size of the first graphic C1 representing the body movement of the subject can be changed more sensitively with respect to the body movement.

### Setting of Boundary Value

The boundary value that is permissible by the MRI apparatus 100 is preferably set according to the imaging conditions such as the imaging target part of the subject 102 by the MRI apparatus 100, the imaging sequence, and the k-space filling method. The processor 210 acquires the imaging conditions from the MRI apparatus 100 to set the boundary value corresponding to the imaging conditions.

### First Example of Setting of Boundary Value

For example, in a head DWI (diffusion weighted imaging) or DTI (diffusion tensor imaging) sequence, it is preferable to set the magnitude of the boundary value corresponding to the second graphic C2 to be smaller than that in other imaging sequences.

In the head DWI or DTI sequence, in a case in which the imaging target part moves between a pair of motion probing gradient (MPG) pulses, an error occurs in calculation of a diffusion coefficient, so that it is desirable to keep the imaging target part as stationary as possible during imaging. Therefore, by reducing the boundary value (by reducing the size of the circle of the second graphic C2 corresponding to the boundary value), it is possible to encourage the subject to reduce the movement of the imaging target part. In addition, in general, the DWI or the DTI sequence is often performed after morphological image capturing using another imaging sequence such as a TIW-based sequence or a T2 W-based sequence. In this case, it is also possible to notify the subject that it is desirable to reduce the movement more than the previous imaging by reducing the boundary value displayed in the other imaging sequence.

### Second Example of Setting of Boundary Value

Even in high-resolution imaging in the orthopedic field such as a knee joint examination, it is desirable to keep the imaging target part as stationary as possible. Therefore, as in the first example, it is possible to notify the subject that it is desirable to reduce the movement of the imaging target part by setting the boundary value to be smaller than that in other imaging sequences.

### Third Example of Setting of Boundary Value

For example, in an MR angiography (MRA) sequence, it is preferable to set the boundary value to be larger than that in other imaging sequences.

In general, the MRA sequence has a long imaging time, and there is no high necessity to keep the imaging target part as compared with a case of capturing a morphological image. Therefore, by increasing the boundary value, a slight movement is permitted. In addition, the subject can be informed that there is no strict tolerance for the movement of the imaging target part.

Staying still for a long time during the imaging time imposes a mental burden on the subject. Notifying the subject of a period in which it is okay to relax has the advantage of reducing the mental burden on the subject.

### Operation Method of Body Movement Display Apparatus

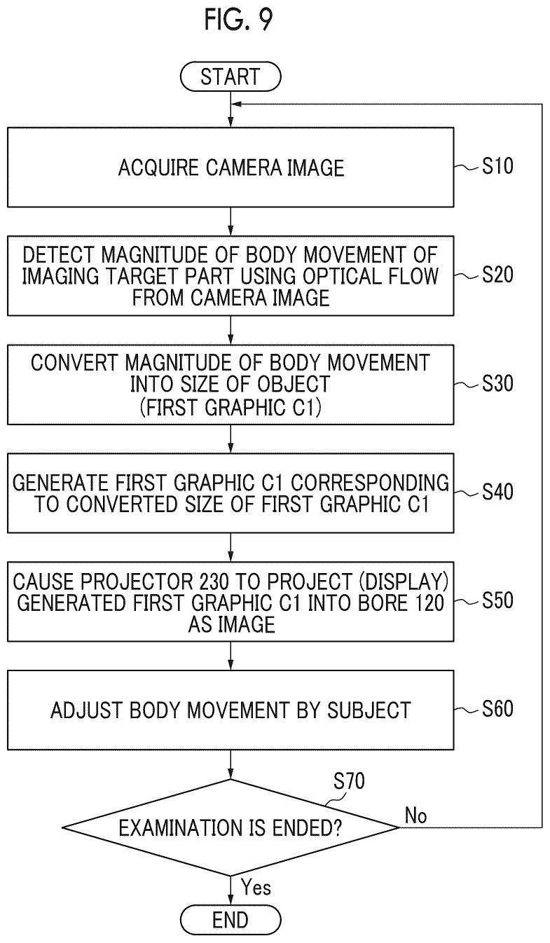

FIG. 9 is a flowchart showing an embodiment of an operation method of the body movement display apparatus according to the embodiment of the present invention, and shows processing contents and processing procedures of the processor 210 of the body movement display apparatus 200 shown in FIG. 4 during the examination of the MRI apparatus 100.

In FIG. 9, in a case in which the examination of the MRI apparatus 100 is started, the processor 210 repeatedly executes processes of step S10 to step S70 until the examination is ended. Here, it is assumed that the processes of step S10 to step S70 are performed in correspondence with a cycle of one frame of the camera image.

The processor 210 starts acquiring the camera image from at least one of the first camera 220A or the second camera 220B that functions as a part of the body movement detection sensor (step S10).

Subsequently, the processor 210 calculates the magnitude of the body movement of the imaging target part of the subject using the optical flow from the acquired camera image (step S20). The processor 210 acquires the body movement vector of the imaging target part between adjacent frames of the camera image, and detects the magnitude of the body movement of the subject as an integrated value (area) of the body movement vector for a certain period of time. Therefore, in step S20, in a case in which a certain period of time has elapsed from the start of the acquisition of the camera image, thereafter, the magnitude of the body movement of the imaging target of the subject is detected (calculated) for each frame.

The processor 210 converts the detected magnitude of the body movement into the size of the object (first graphic C1) (step S30). In this case, the processor 210 converts the magnitude of the body movement into the size of the first graphic C1 according to parameters of a linear conversion or a non-linear conversion set in advance (see graphs in FIGS. 6 and 7). In addition, in this conversion, it is preferable to perform a conversion weighted based on the imaging conditions such as the imaging target part, the imaging sequence, and the k-space filling method. This is because the influence of the body movement of the subject on the imaging of the MRI apparatus 100 varies depending on the imaging conditions.

Next, the processor 210 generates the first graphic C1 corresponding to the converted size of the first graphic C1 (step S40). Since the first graphic C1 of this example is a graphic having a circular outer shape, the converted size of the first graphic C1 corresponds to the diameter of the circle or the area of the circle.

The processor 210 causes the projector 230 to project (display) the generated first graphic C1 into the bore 120 as an image (step S50). In this case, it is preferable that the processor 210 also simultaneously displays the second graphic C2 corresponding to the boundary value of the magnitude of the body movement of the subject 102. The second graphic C2 is a concentric circle having the same center as the first graphic C1 (see FIG. 5).

The subject can grasp the magnitude of his/her own body movement by observing the size of the first graphic C1 included in the image displayed in the bore 120, and can adjust (suppress) the body movement as necessary (step S60).

Subsequently, the processor 210 determines whether or not the examination of the MRI apparatus 100 is ended (step S70). Then, in a case in which it is determined that the examination of the MRI apparatus 100 has not been ended (examination is in progress) (in a case of “No”), the process proceeds to step S10, and the processes of step S10 to step S70 are repeated. Since the processes from step S10 to step S70 are performed for each cycle of one frame as described above, the first graphic C1 corresponding to the magnitude of the current body movement of the subject is displayed in real time on the image projected into the bore 120, and the subject can grasp the magnitude of his/her own body movement in real time by viewing the projected image.

On the other hand, in step S70, in a case in which it is determined that the examination of the MRI apparatus 100 has been ended (in a case of “Yes”), the operation of the body movement display apparatus 200 is ended. The processor 210 can determine whether or not the imaging has been ended based on the communication with the controller 150 of the MRI apparatus 100.

According to the operation method of the body movement display apparatus according to the embodiment of the present invention, the subject 102 can check the magnitude of his/her own body movement by viewing the image of the first graphic C1 and the like projected into the bore 120. In particular, the size (diameter, area) of the first graphic C1 changes according to the magnitude of the body movement of the subject 102, but, since the outer shape of the first graphic C1 about a fixed center is a circle, the subject 102 can visually recognize the first graphic C1 whose size changes without moving the line of sight, and the movement of the eyes can be minimized. As a result, it is possible to reduce the induction of the movement of the head accompanied by the movement of the eyes and the movement of parts other than the head.

### Second Embodiment of Body Movement Display Apparatus

Next, a second embodiment of the body movement display apparatus according to the present invention will be described.

In the examination of the abdomen of the subject using the MRI apparatus 100, a respiratory-gated imaging method or a breath-hold imaging method is applied in order to reduce motion artifacts caused by the subject's breathing.

In the respiratory-gated imaging method or the like, the respiratory bands 222A and 222B shown in FIG. 3 are attached to the abdomen of the subject, and data is measured only in a state in which the movement of the abdomen is small and stable (mainly during expiration).

The second embodiment of the body movement display apparatus is different from the first embodiment in that, in a case in which the MRI apparatus 100 executes the respiratory-gated imaging method or the like, a display form of the image projected from the projector 230 is changed between the respiratory-gated measurement period and the non-measurement period.

As an example of changing the display form between the image during the respiratory-gated measurement and the image during the non-measurement, the following method is considered.

(1) The brightness of the entire graphic or image to be displayed is changed. For example, the display is made brighter in the data acquisition period than in the data non-acquisition period to encourage the subject 102 to suppress the body movement. Since the brightness of the display is changed to notify that the respiratory-gated measurement is being performed or not being performed, it is possible to suppress the movement of the line of sight of the subject 102 as compared with a case of providing a notification with characters.

(2) The brightness is gradually changed (faded in) starting a few seconds before the data acquisition period and reaching the level of brightness used during the data acquisition period. As a result, it is possible to give a warning to the subject 102 to start the data acquisition.

(3) The color of the displayed graphic is changed. The color change includes displaying in color during the respiratory-gated measurement and in black-and-white during the non-measurement.

According to the second embodiment, it is possible to notify the subject 102 of the imaging period, and the subject 102 can suppress the body movement (respiratory motion) while viewing the image during the imaging period.

### Third Embodiment of Body Movement Display Apparatus

The processor 210 of a third embodiment of the body movement display apparatus is different from the first embodiment in that a warning is issued in a case in which the size of the first graphic C1 approaches the boundary value corresponding to the magnitude of the body movement of the subject that is permissible by the MRI apparatus 100 (in a case in which the size of the first graphic C1 approaches the boundary value beyond a threshold value) or exceeds the boundary value.

The threshold value for determining whether or not the magnitude of the body movement of the subject has approached the boundary value can be set to about 0.8 of the magnitude of the boundary value, but may be set as appropriate.

The warning can be issued by one or more of a warning sound generator, a display, an illumination device in the gantry of the MRI apparatus 100, and a vibration generator.

The warning sound generator generates a beep sound or the like to notify the subject that the magnitude of the body movement is approaching the boundary value. The display including the projector 230 notifies the subject that the magnitude of the body movement is approaching the boundary value, for example, by changing the graphic (blinking the second graphic C2 corresponding to the boundary value). In addition, the illumination device in the gantry notifies the subject that the magnitude of the body movement is approaching the boundary value by illuminating the light in the gantry.

In addition, the vibration generator notifies the subject that the magnitude of the body movement is approaching the boundary value by vibrating a vibrating body gripped by the subject, for example. In this case, the level of the warning can be changed by changing the frequency of the vibration, and the frequency can be increased as the boundary value is approached, and the attention can be called.

The warning sound generator, the display, the illumination device in the gantry of the MRI apparatus 100, and the vibration generator may be appropriately combined to issue the warning.

### Fourth Embodiment of Body Movement Display Apparatus

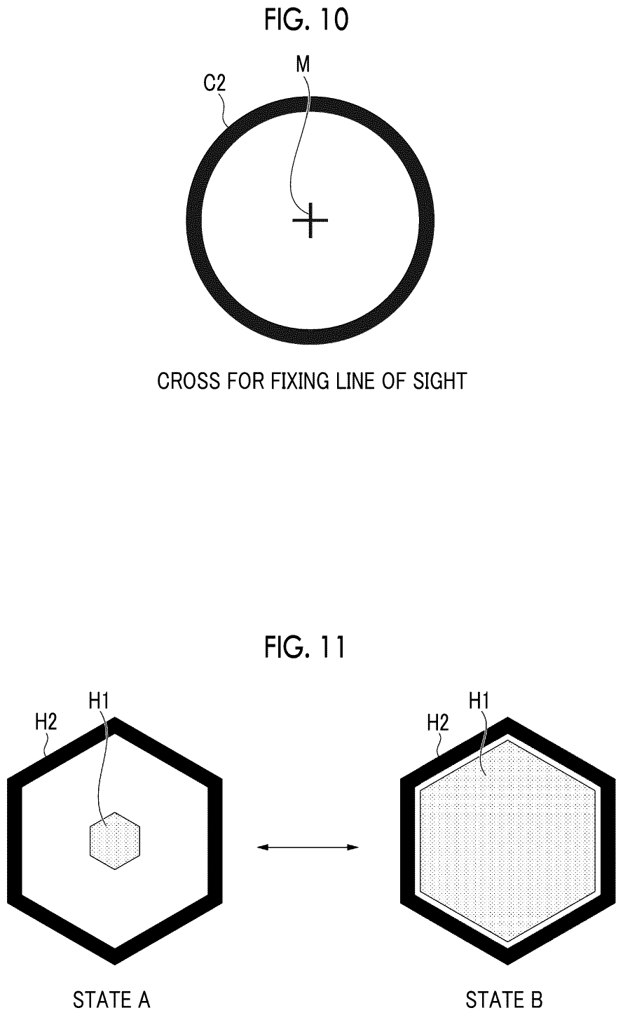

FIG. 10 is a diagram showing a part of an image projected by the projector 230, and particularly shows a case in which a mark for fixing a line of sight is displayed.

The processor 210 of a fourth embodiment of the body movement display apparatus displays a mark M for fixing a line of sight at the center of the first graphic C1 shown in FIG. 5. The mark M of this example is a cross mark, but the present invention is not limited thereto.

Although the first graphic C1 shown in FIG. 5 is also displayed, the first graphic C1 is omitted in FIG. 10. In addition, it is preferable that the mark M is combined on the first graphic C1 as a mark M having a color and/or brightness different from the first graphic C1 so that the mark M is always visible even in a case in which the first graphic C1 is displayed.

According to the fourth embodiment, the subject 102 can be more effectively encouraged to fix his/her line of sight in a case of visually recognizing the first graphic C1 or the like, and a line-of-sight movement that induces the body movement including the movement of the head can be prevented.

### Others

The graphic including the first graphic C1 of the present embodiment has a circular outer shape, but the present invention is not limited thereto. The graphic may be a graphic that is rotationally symmetric about a fixed center, for example, a regular polygon.

FIG. 11 is a diagram showing a graphic projected by the projector 230, and is a diagram showing a graphic obtained by combining a first graphic H1 that changes according to the magnitude of the body movement and a second graphic H2 corresponding to the magnitude of the boundary value.

The outer shape of the first graphic H1 shown in FIG. 11 is a regular hexagon, and similarly, the second graphic H2 is also a regular hexagon, and the centers of the first graphic H1 and the second graphic H2 coincide with each other. The first graphic H1 corresponds to the first graphic C1 shown in FIG. 5, and the second graphic H2 corresponds to the second graphic C2 shown in FIG. 5, and both graphics have different outer shapes.

The first graphic H1 corresponds to the first graphic C1 shown in FIG. 5, and the first graphic H1 on a left side of FIG. 11 indicates a state A in which the body movement is sufficiently small, and the first graphic H1 on a right side of FIG. 11 indicates a state B in which the body movement is large (approaching the second graphic H2 of the boundary value)

In addition, in the present embodiment, the display that displays the first graphic or the like is the projector 230 that projects an image onto the bore 120 in the gantry 110 of the MRI apparatus 100, but the present invention is not limited to this, and, for example, a monitor such as a head-up display, a head-mounted display, a liquid crystal display in the bore 120, or an organic EL display, a monitor outside the bore 120, and a set of a mirror for viewing the monitor can be considered.

Further, the image diagnostic apparatus to which the body movement display apparatus is applied is not limited to the MRI apparatus, and may be, for example, an X-ray CT apparatus.

Furthermore, in the present embodiment, each process is executed by any computer. In addition, any computer may execute these processes by a processor, a program, or a combination thereof. Any computer may be a general-purpose computer, a computer for a specific use, a system such as a workstation, or other hardware elements capable of executing a program.

The processor may be configured by one or more pieces of hardware, and the type of hardware is not limited. For example, the processor can be configured with a central processing unit (CPU), a micro processing unit (MPU), a programmable logic device such as a field programmable gate array (FPGA), a dedicated circuit for executing specific processing such as an application specific integrated circuit (ASIC), or hardware such as a graphic processing unit (GPU) or a neural processing unit (NPU). In addition, the processor has each unit or each means that executes various types of processes in the present embodiment. In addition, the types of hardware may be a combination of different types of hardware. In a case in which a plurality of pieces of hardware are configured to execute one or a plurality of processes of a certain processor, the plurality of pieces of hardware may be present in devices physically separated from each other, or may be present in the same device. In addition, in any of the embodiments, the order of each process executed by the processor is not limited to the above order and may be changed as appropriate. The hardware is configured by an electric circuit (circuitry) in which circuit elements such as semiconductor elements are combined.

Further, the present embodiment may be realized by hardware, software, firmware, microcode, or a combination thereof. Software, firmware, and microcode are configured by a program. In addition, the program may be, for example, a program module group, and each function thereof may be realized by a processor configured to execute each function. The program may be a program code or a plurality of code segments stored in one or a plurality of non-transitory computer-readable media (for example, a storage medium or other storage). The program may be divided and stored in a plurality of non-transitory computer-readable media present in devices physically separated from each other. The program code or the code segment may represent any combination of a procedure, a function, a subprogram, a routine, a subroutine, a module, a software package, a class, an instruction, a data structure, or a program statement. The program code or the code segment may be connected to another code segment or a hardware circuit by transmitting and receiving information, data, an argument, a parameter, or memory contents.

The present invention is not limited to the embodiments described above, and various modifications can be made without departing from the spirit of the present invention.

### EXPLANATION OF REFERENCES

100: MRI apparatus
102: subject
104: static magnetic field generating magnet
106: gradient magnetic field coil
108: transmission coil
110: gantry
112: high-frequency magnetic field generator
114: receiver
116: gradient magnetic field power supply
118: signal processing unit
120: bore
130: examination bed
130A: top plate
140: receive coil
142: signal cable
150: controller
160: operation unit
170: display
200: body movement display apparatus
210: processor
220A: first camera
220B: second camera
222A: respiratory band
230: projector
C1. H1: first graphic
C2. H2: second graphic
Im: image
M: marker
S10 to S70: step showing operation of body movement display apparatus

What is claimed is:

1. A body movement display apparatus comprising:
a processor;
a display that displays an image in a manner that is visible to a subject during an examination of the subject using an image diagnostic apparatus; and
a body movement detection sensor that detects a body movement of the subject,
wherein the processor
generates a first graphic that is rotationally symmetric about a fixed center and whose size changes according to a magnitude of the body movement of the subject detected by the body movement detection sensor, and
causes the display to display the generated first graphic as the image.

2. The body movement display apparatus according to claim 1,
wherein the processor converts the magnitude of the body movement of the subject into the size of the first graphic, and generates the first graphic corresponding to the converted size of the first graphic.

3. The body movement display apparatus according to claim 2,
wherein the conversion of the magnitude of the body movement of the subject into the size of the first graphic is a linear conversion or a non-linear conversion.

4. The body movement display apparatus according to claim 2,
wherein the conversion of the magnitude of the body movement of the subject into the size of the first graphic is a conversion weighted based on a magnitude of an influence of the body movement of the subject on imaging of the image diagnostic apparatus.

5. The body movement display apparatus according to claim 4,
wherein the magnitude of the influence of the body movement of the subject on the imaging of the image diagnostic apparatus changes depending on at least one of an imaging target part of the subject imaged by the image diagnostic apparatus, an imaging sequence in the image diagnostic apparatus, or a k-space filling method used in the image diagnostic apparatus.

6. The body movement display apparatus according to claim 1,
wherein a boundary value of the magnitude of the body movement of the subject that is permissible by the image diagnostic apparatus and that presents a relation with the size of the first graphic is set, and
the processor causes the display to display a second graphic having a size corresponding to the boundary value and having a similar outer shape to the first graphic such that a center of the second graphic coincides with the center of the first graphic.

7. The body movement display apparatus according to claim 6,
wherein the boundary value is set by at least one of an imaging target part of the subject imaged by the image diagnostic apparatus, an imaging sequence in the image diagnostic apparatus, or a k-space filling method used in the image diagnostic apparatus.

8. The body movement display apparatus according to claim 6,
wherein the processor causes the display to display the first graphic and the second graphic such that the first graphic and the second graphic differ in at least one of a color, a line type, or brightness.

9. The body movement display apparatus according to claim 6,
wherein the processor issues a warning in a case in which the size of the first graphic approaches the boundary value beyond a threshold value or exceeds the boundary value.

10. The body movement display apparatus according to claim 9,
wherein the warning is issued by one or more of a warning sound generator, the display, an illumination device in a gantry of the image diagnostic apparatus, and a vibration generator.

11. The body movement display apparatus according to claim 1,
wherein the body movement detection sensor includes a camera that images the subject and that outputs an image of the subject, and an image processing unit that processes the image to detect the body movement of the subject, and
the image processing unit
extracts an imaging target part of the subject included in the image, the imaging target part being imaged by the image diagnostic apparatus,
acquires a movement of the extracted imaging target part between consecutive frames of the image as a body movement vector of the imaging target part, and
detects the magnitude of the body movement of the subject from the body movement vector.

12. The body movement display apparatus according to claim 1,
wherein the first graphic has an outer shape of a circle or a regular polygon.

13. An image diagnostic system comprising:
an image diagnostic apparatus; and
the body movement display apparatus according to claim 1.

14. The image diagnostic system according to claim 13,
wherein the image diagnostic apparatus includes a magnetic resonance imaging apparatus or an X-ray CT apparatus.

15. An operation method of a body movement display apparatus including a processor, a display that displays an image in a manner that is visible to a subject during an examination of the subject using an image diagnostic apparatus, and a body movement detection sensor that detects a body movement of the subject, the operation method comprising:
a step of, via the processor, acquiring a magnitude of the body movement of the subject from the body movement detection sensor;
a step of, via the processor, generating a first graphic that is rotationally symmetric about a fixed center and whose size changes according to the acquired magnitude of the body movement of the subject; and
a step of, via the processor, causing the display to display the generated first graphic as the image.
